(19) United States
(12) Patent Application Publication
(10) Pub. No.: US 2025/0120331 A1
(43) Pub. Date: Apr. 17, 2025

(54) Systems and Methods for Negative Pressure Grain Imbibition

(71) Applicant: Smart Sprouts International LLC, Carpenter, WY (US)

(72) Inventor: Shawn D. Jenkins, Cheyenne, WY (US)

(73) Assignee: Smart Sprouts International LLC, Carpenter, WY (US)

(21) Appl. No.: 18/669,127

(22) Filed: May 20, 2024

Related U.S. Application Data
(60) Provisional application No. 63/511,128, filed on Jun. 29, 2023.

(51) Int. Cl. A01C 1/00

(52) U.S. Cl. CPC A01C 1/00

(57) ABSTRACT

The present disclosure relates in general to the imbibition of grains for the agronomic, horticulture, animal feed, and malting industries. More specifically, the present disclosure relates to systems and methods for overcoming individual differences in grain morphology using negative pressure to achieve a uniform rate at which grains absorb water, nutrients, and additives from the surrounding environment to increase the productivity, efficiency, and economics of industries that rely on grain germination and seedling development to produce a product. The application of negative pressure to grains in aqueous solutions containing plant additives such as phytohormones has been found to reduce seedling-to-seedling developmental variability, hydration variability, and the concentration of phytohormones needed to achieve improved germination rates, the mobilization of starches, and the uniform development rate of seedlings.

### CROSS-REFERENCE TO RELATED APPLICATIONS

This application claims priority under 35 U.S.C. § 119 to U.S. Provisional Patent Application No. 63/511,128 filed Jun. 29, 2023, herein incorporated by reference in its entirety.

### FIELD OF THE INVENTION

The present disclosure relates in general to the imbibition of grains for the agronomic, horticulture, animal feed, and malting industries. More particularly, but not exclusively, the present disclosure relates to systems and methods for overcoming individual differences in grain morphology using negative pressure to achieve a uniform rate at which grains absorb water, nutrients, and additives from the surrounding environment to increase the productivity, efficiency, and economics of industries that rely on grain germination and seedling development to produce a product.

### BACKGROUND

A grain comprises an embryo packaged along with a food supply, called endosperm, inside a protective seed coat. Germination of the grain largely depends on two critical environmental factors: temperature and imbibition. Imbibition is the absorption of water by a dry seed due to its lower water potential as compared to the higher water potential of the surrounding environment. Water is known to move from a higher potential to a lower potential at varying speeds. The speed at which water travels down water potential gradients, or here absorption through the seed coat, may be impacted by a variety of contributing factors. Some of these contributing factors include individual grain morphologies, such as size, shape, hardness, and composition. Mitigating the impact of these individual grain morphologies is desired, as successful imbibition is required before the initiation of metabolic processes, triggering the release of phytohormones from the embryo and the onset of germination.

Phytohormones are plant hormones produced in extremely low concentrations which control all aspects of plant growth and development. One important example of a phytohormone is gibberellin, which acts as a signal for the grain to begin synthesizing and secreting digestive enzymes that hydrolyze its stored food supply, often comprising starch, proteins, and lipids. These digestive enzymes include α-amylase, an enzyme that begins to hydrolyze starch into small, soluble molecules such as oligosaccharides, fructose, maltose and glucose, which are consumed during growth of the embryo as it transitions into a seedling. While α-amylase is the most important and well-studied enzyme, other enzymes such as maltase and glucosidase are also necessary to fully hydrolyze starch to glucose for ultimate use by the plant seedling.

In commercial applications nutrients, additives, fungicides, insecticides, signaling compounds, and exogenous phytohormones (collectively, “plant additives”), are often added to the water and/or the surrounding environment of grains to improve germination rates, the mobilization of starches, and the development rate of seedlings. See Hedden et al., A Century Of Gibberellin Research, JOURNAL OF PLANT GROWTH REGULATION, Vol. 34, pp. 740-760 (Oct. 13, 2015). The utility of such commercial applications however is severely limited due to the variability in water imbibition rates caused by individual differences in grain morphologies. See Palmer, G. H., The Industrial Use Of Gibberellic Acid And Its Scientific Basis—A Review, JOURNAL OF THE INSTITUTE OF BREWING, Vol. 80, Iss. 1, pp. 13-30 (January-February, 1974); see also Briggs, D. E., Accelerating Malting: A Review of Some Lessons of the Past from the United Kingdom, JOURNAL OF THE AMERICAN SOCIETY OF BREWING CHEMISTS, Vol. 45, pp. 1-6 (Feb. 6, 2018). Indeed the commercial application of additives to the water and/or the surrounding environment of grain, particularly exogenous phytohormones, have proven exceedingly difficult to accurately dose because of the grain's narrow range of responsiveness (i.e., 1.5-3.0 ppm) in combination with extremely low concentrations needed to yield a positive result (e.g., 2.0 ppm). See Briggs, D. E., Accelerating Malting: A Review of Some Lessons of the Past from the United Kingdom, JOURNAL OF THE AMERICAN SOCIETY OF BREWING CHEMISTS, Vol. 45, pp. 1-6 (Feb. 6, 2018). As a result, individual differences in grain morphologies introduce significant and undesirable variations in water and plant additive imbibition rates that negatively impact germination times and resultant seedling development across the agronomic, horticulture, animal feed, and malting industries.

Thus a desire remains to develop systems and methods that mitigate variations in water and plant additive imbibition rates for grains that negatively impact germination times and resultant seed development across the agronomic, horticulture, animal feed, and malting industries. A desire further remains to develop systems and methods that permit accurate dosing of plant additives, and particularly exogenous phytohormones, to the water and/or the surrounding environment of grains to maximize positive results without contributing to nutrient pollution or waste. A desire still further remains to develop systems and methods that overcome individual differences in grain morphology to achieve a uniform rate at which grains absorb water and plant additives from the surrounding environment to increase the productivity, efficiency, and economics of industries that rely on grain germination and seedling development to produce a product.

### SUMMARY

In one aspect of the present disclosure, a method for increasing the effects of phytohormones on grains is provided. The method may include forming an aqueous solution having a chosen concentration of at least one phytohormone in water. The method may further include contacting the grains with the solution, reducing pressure over the grains and the solution, and maintaining the reduced pressure for a certain period of time.

In another aspect of the present disclosure, a method for overcoming differences in grain morphology using negative pressure is provided. The method may include providing a system for imbibing grains using negative pressure. The method may further include introducing the grains to an aqueous solution comprising at least one plant additive in water, and an optional reactive oxygen species. The grains in solution may be placed into a vacuum chamber and the pressure reduced to approximately 10.0-25.0 inches of mercury (“Hg”), wherein the reduced pressure may be maintained for approximately 60-240 minutes. After imbibition, the grains may be removed from the vacuum chamber and the solution and introduced into a growing station where they are developed into plant seedlings and ultimately harvested for their intended use in, e.g., the agronomic, horticulture, animal feed, and malting industries.

In another aspect of the present disclosure, a system for imbibing grains using negative pressure to overcome differences in grain morphology is provided. The system may include a plurality of grains to be imbibed, an aqueous solution for imbibing the grains containing at least one plant additive in water, a vacuum chamber configured to apply negative pressure to the grains in the solution, and a growing station for cultivating imbibed grains into plant seedlings.

### Objects, Features, and Advantages

It is a principal object, feature, and advantage of the present disclosure to overcome the aforementioned deficiencies in the art and provide systems and methods that mitigate undesirable variations in water and plant additive imbibition rates for grains that negatively impact germination times and seedling development across the agronomic, horticulture, animal feed, and malting industries.

Another object, feature, and advantage of the present disclosure is to provide systems and methods that permit accurate dosing of plant additives, and particularly exogenous phytohormones, to the water and/or the surrounding environment of grains to maximize positive results without contributing to nutrient pollution or waste.

Yet another object, feature, and advantage of the present disclosure is to provide systems and methods that overcome individual differences in grain morphology to achieve a uniform rate at which grains absorb water and plant additives from the surrounding environment to increase the productivity, efficiency, and economics of industries that rely on grain germination and seedling development to produce a product.

A further object, feature, and advantage of the present disclosure is to provide systems and methods that maximize the rate at which grains imbibe water and plant additives for improved germination rates, the mobilization of starches, and the uniform development rate of seedlings.

A still further object, feature, and advantage of the present disclosure is to provide systems and methods that improve the uniformity at which grains imbibe water and plant additives to reduce variability in germination rates, the mobilization of starches, and the uniform development rate of seedlings.

Another object, feature, and advantage of the present disclosure is to provide systems and methods that ensure individual grains are imbibed at approximately the same rate to allow for accurate and precise dosing of plant additives, and particularly exogenous phytohormones, required to achieve uniform seedling response.

Yet another object, feature, and advantage of the present disclosure is to provide systems and methods that improve the uniformity at which individual grains imbibe water and plant additives to increase yields, improve nutritional value, and reduce costs for the agronomic, horticulture, animal feed, and malting industries.

A further object, feature, and advantage of the present disclosure is to provide systems and methods that advance the effectiveness of seed treatment of insecticides and fungicides as compared to traditional surface coating procedures for agronomic applications.

A still further object, feature, and advantage of the present disclosure is to provide systems and methods that efficiently and effectively prime seeds for high value crops to maximize individual seed response and production in horticulture applications. Another object, feature, and advantage of the present disclosure is to provide systems and methods that enable more efficient, profitable, and sustainable animal protein production in animal feeding applications, whereby enteric methane emissions from ruminants are reduced.

A further object, feature, and advantage of the present disclosure is to provide systems and methods that improve the rate and ratio at which input substrates are transformed into usable fermentation byproducts in malting applications, whereby wastes associated with the fermentation process such as carbon dioxide and methane are reduced.

A still further object, feature, and advantage of the present disclosure is to provide systems and methods that reduce greenhouse gas emissions while offering opportunities for offset or inset carbon credit generation.

Other objects, features, or advantages of this disclosure will become apparent from the following detailed description and claims, taken in conjunction with the accompanying drawings that set forth, by way of illustration and example and without limitation, certain aspects of this disclosure. No single aspect need provide each and every object, feature, or advantage. Thus the present disclosure is not to be limited to or by these objects, features, and advantages.

### BRIEF DESCRIPTION OF THE DRAWINGS

The accompanying drawings, incorporated herein and forming a part of the specification, illustrate aspects of the present disclosure together with the detailed description and claims.

FIG. 1 is a diagram illustrating a system of using negative pressure to imbibe grains according to the present disclosure.

FIG. 2 is a flowchart illustrating a method of using negative pressure to imbibe grains according to a first aspect of the present disclosure.

FIG. 2A is a pictorial representation of seedling development after 72 hours in the growing station for grains imbibed using negative pressure according to the method of FIG. 2.

FIG. 2B is a pictorial representation of seedling development after 72 hours in the growing station for grains not imbibed using negative pressure according to the method of FIG. 2.

FIG. 3 is a graph showing glucose concentration as a percentage of dry matter weight at atmospheric pressure (curve (a)) and at a vacuum of 25 in. of Hg (curve (b)), both for 180 min., for Winner hard red winter seedlings in an aqueous solution with no exogenous gibberellic acid (GA₃) as compared to Winner hard red winter seedlings in an aqueous solution with 3 ppm of exogenous gibberellic acid (GA₃) and Winner hard red winter seedlings in an aqueous solution with 10 ppm of exogenous gibberellic acid (GA₃).

FIG. 4 is a graph showing maltose concentration as a percentage of dry matter weight at atmospheric pressure (curve (a)) and at a vacuum of 25 in. of Hg (curve (b)), both for 180 min., for Winner hard red winter seedlings in an aqueous solution with no exogenous gibberellic acid (GA₃) as compared to Winner hard red winter seedlings in an aqueous solution with 3 ppm of exogenous gibberellic acid (GA₃) and Winner hard red winter seedlings in an aqueous solution with 10 ppm of exogenous gibberellic acid (GA₃).

FIG. 5 is a graph showing fructose concentration as a percentage of dry matter weight at atmospheric pressure (curve (a)) and at a vacuum of 25 in. of Hg (curve (b)), both for 180 min., for Winner hard red winter seedlings in an aqueous solution with no exogenous Gibberellic Acid (GA₃) as compared to Winner hard red winter seedlings in an aqueous solution with 3 ppm of exogenous gibberellic acid (GA₃) and Winner hard red winter seedlings in an aqueous solution with 10 ppm of exogenous gibberellic acid (GA₃).

FIG. 6 is a graph showing total sugar concentration as a percentage of dry matter weight at atmospheric pressure (curve (a)) and at a vacuum of 25 in. of Hg (curve (b)), both for 180 min., for Winner hard red winter seedlings in an aqueous solution with no exogenous gibberellic acid (GA₃) as compared to Winner hard red winter seedlings in an aqueous solution with 3 ppm of exogenous gibberellic acid (GA₃) and Winner hard red winter seedlings in an aqueous solution with 10 ppm of exogenous gibberellic acid (GA₃).

FIG. 7 is a graph showing germination percentage at atmospheric pressure (curve (a)) and at a vacuum of 25 in. of Hg (curve (b)), both for 180 min., for Winner hard red winter seedlings in an aqueous solution with no exogenous gibberellic acid (GA₃) as compared to Winner hard red winter seedlings in an aqueous solution with 3 ppm of exogenous gibberellic acid (GA₃) and Winner hard red winter seedlings in an aqueous solution with 10 ppm of exogenous gibberellic acid (GA₃).

FIG. 8 is a graph showing germination percentage for Winner hard red winter seedlings in aqueous solutions at atmospheric pressure (curves (a)), and at 20 in. of Hg (curves (b)), for 180 min., containing:
(1) no exogenous phytohormones (control);
(2) gibberellic acid (GA₃) at 0.75 ppm, 1.5 ppm, 3, 6 ppm, and 10 ppm;
(3) hydrogen peroxide (H₂O₂) at 600 ppm and 1200 ppm;
(4) 1-Naphthaleneacetamide at 1 ppm; and
(5) Thidiazuron at 1 ppm and 5 ppm.

FIG. 9 is a graph showing coleoptile lengths in mm for Winner hard red winter seedlings in aqueous solutions at atmospheric pressure (curves (a)), and at 20 in. of Hg (curves (b)), for 180 min., containing:
(1) no exogenous phytohormones (control);
(2) gibberellic acid (GA₃) at 0.75 ppm, 1.5 ppm, 3, 6 ppm, and 10 ppm;
(3) hydrogen peroxide (H₂O₂) at 600 ppm and 1200 ppm;
(4) 1-Naphthaleneacetamide at 1 ppm; and
(5) Thidiazuron at 1 ppm and 5 ppm.

FIG. 10 is a graph showing radicle lengths in mm for Winner hard red winter seedlings in aqueous solutions at atmospheric pressure (curves (a)), and at 20 in. of Hg (curves (b)), for 180 min., containing:
(1) no exogenous phytohormones (control);
(2) gibberellic acid (GA₃) at 0.75 ppm, 1.5 ppm, 3, 6 ppm, and 10 ppm;
(3) hydrogen peroxide (H₂O₂) at 600 ppm and 1200 ppm;
(4) 1-Naphthaleneacetamide at 1 ppm; and
(5) Thidiazuron at 1 ppm and 5 ppm.

FIG. 11 is a graph showing coleoptile+radicle lengths in mm for Winner hard red winter seedlings in aqueous solutions at atmospheric pressure (curves (a)), and at 20 in. of Hg (curves (b)), for 180 min., containing:
(1) no exogenous phytohormones (control);
(2) gibberellic acid (GA₃) at 0.75 ppm, 1.5 ppm, 3, 6 ppm, and 10 ppm;
(3) hydrogen peroxide (H₂O₂) at 600 ppm and 1200 ppm;
(4) 1-Naphthaleneacetamide at 1 ppm; and
(5) Thidiazuron at 1 ppm and 5 ppm.

FIG. 12 is a graph showing biomass pixels for Winner hard red winter seedlings in aqueous solutions at atmospheric pressure (curves (a)), and at 20 in. of Hg (curves (b)), for 180 min., containing:
(1) no exogenous phytohormones (control);
(2) gibberellic acid (GA₃) at 0.75 ppm, 1.5 ppm, 3, 6 ppm, and 10 ppm;
(3) hydrogen peroxide (H₂O₂) at 600 ppm and 1200 ppm;
(4) 1-Naphthaleneacetamide at 1 ppm; and
(5) Thidiazuron at 1 ppm and 5 ppm.

FIG. 13 is a graph showing moisture percentage for Winner hard red winter seedlings in aqueous solutions containing no exogeneous phytohormones held at a constant 15 in. of Hg (curve (a)) and a constant 25 in. of Hg (curve (b)), each for 60 min. (Constant); as compared to Winner hard red winter seedlings in aqueous solutions containing no exogeneous phytohormones held at a constant 15 in. of Hg (curve (a)) and a constant 25 in. of Hg (curve (b)), each for 60 min. with atmospheric pressure being restored and vacuum reapplied every 15 min. (Alternate); as further compared to Winner hard red winter seedlings in aqueous solutions containing no exogeneous phytohormones held at a constant 15 in. of Hg (curve (a)) and a constant 25 in. of Hg (curve (b)), each for 60 min. with vibration being applied to the vacuum chamber holding the seedlings and hydrating solution for 1 min. every 15 min. (Vibration).

FIG. 14 is a pictorial representation of cross sections of grains showing endosperm hydration at varying levels of positive pressure grain imbibition as compared to control.

FIG. 15 is a graph comparing germination percentages for grains imbibed with positive pressure, negative pressure, as compared to control.

### DETAILED DESCRIPTION

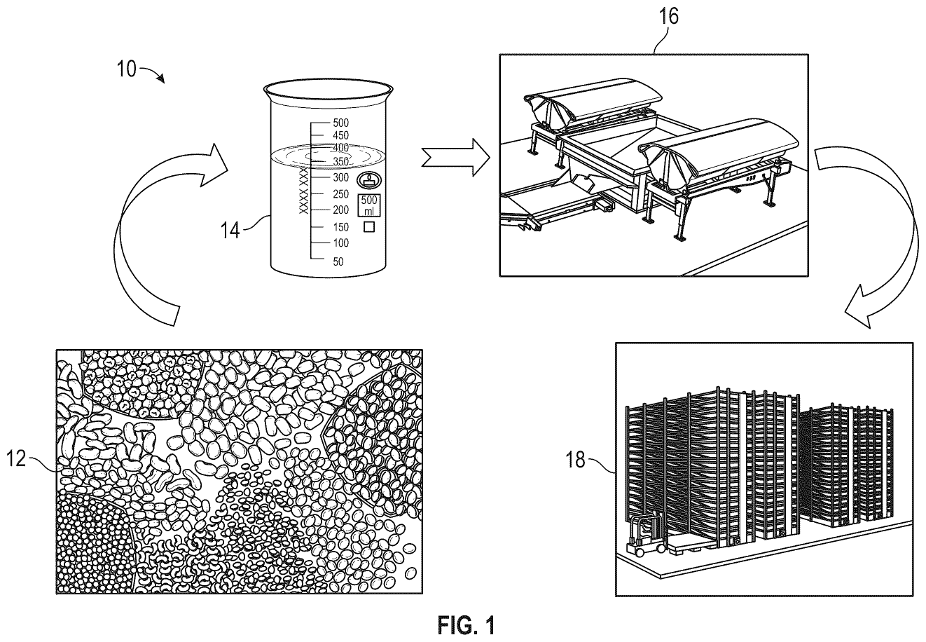

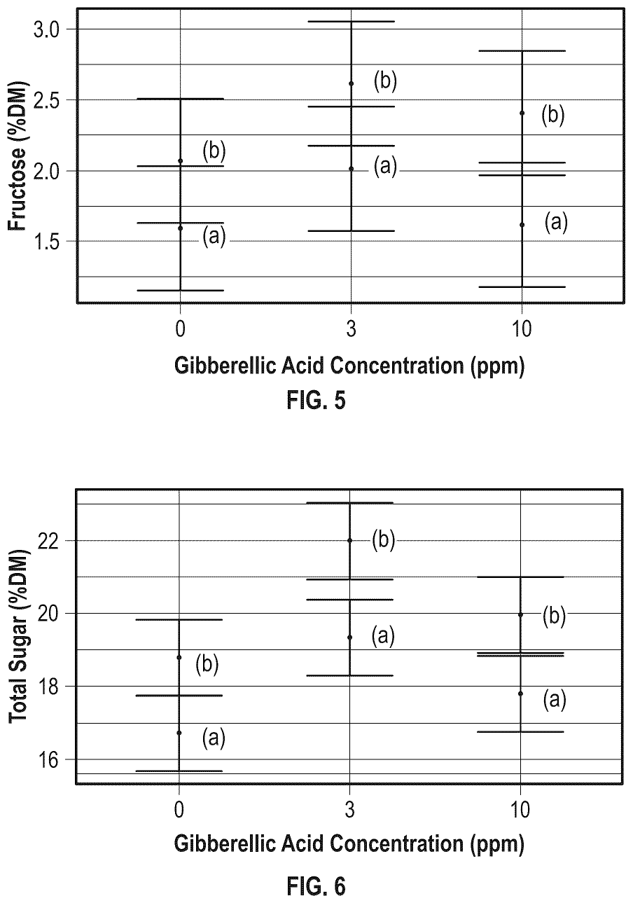

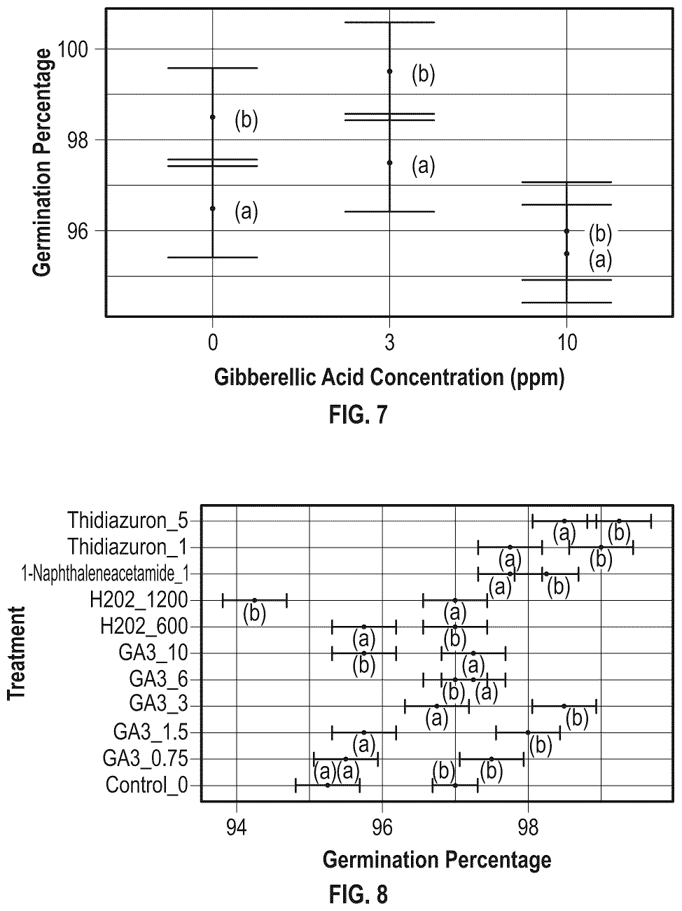

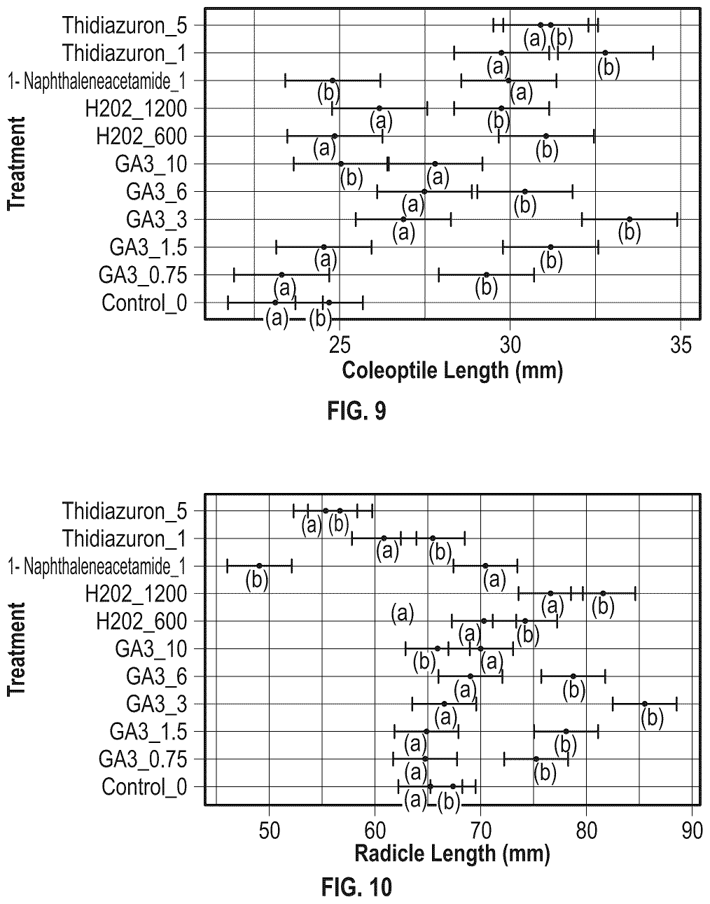

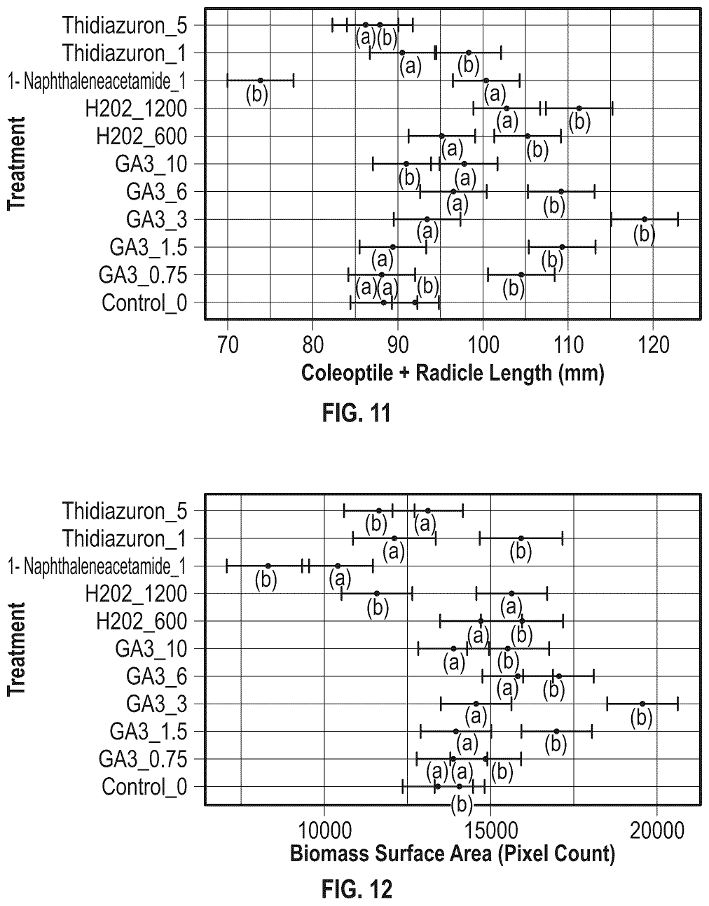

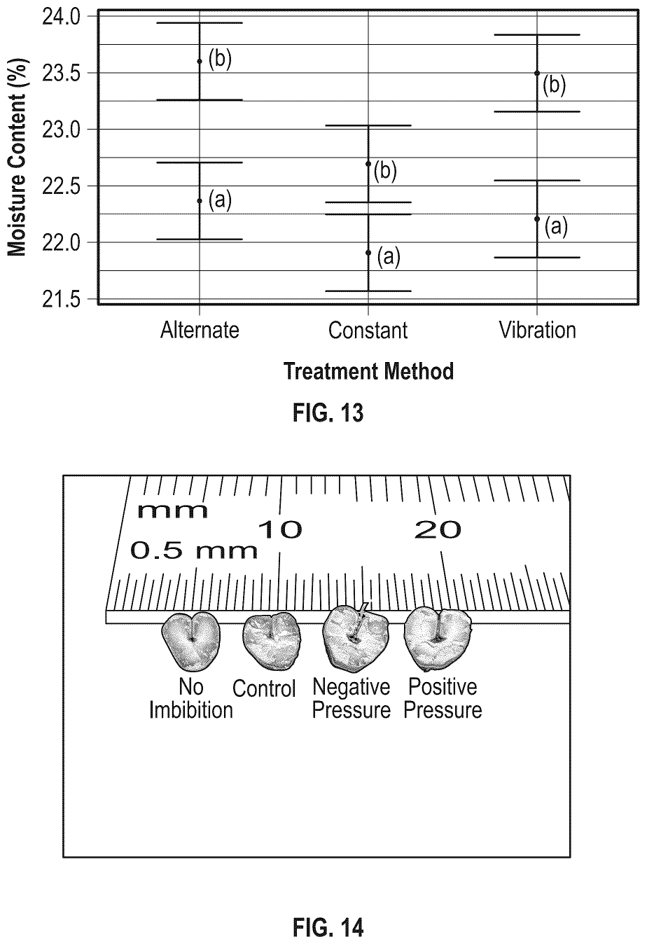

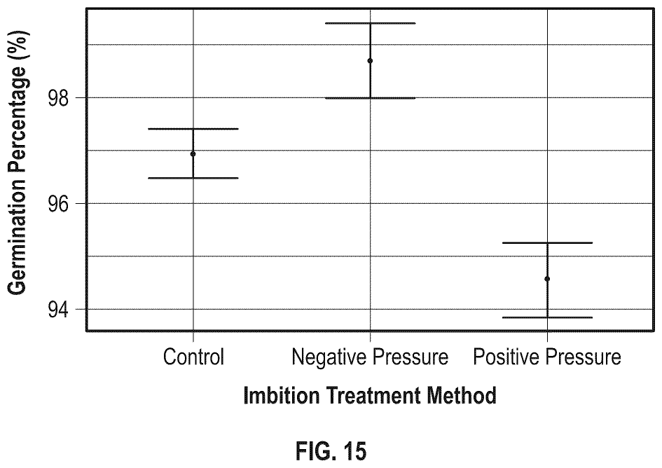

Referring generally to FIGS. 1-15, the present disclosure relates to systems and methods that utilize negative pressure to overcome undesirable variations in water and plant additive imbibition rates caused by individual differences in grain morphology. Mitigating such variations provide significant benefits to the agronomic, horticulture, animal feed, and malting industries. Some of these benefits include, but are not limited to, the ability to accurately dose plant additives, and particularly exogenous phytohormones, to the water and/or the surrounding environment of grains to maximize rates of imbibition. Maximizing rates of grain imbibition provide for improved germination rates, the mobilization of starches, and the uniform development rate of seedlings. An additional benefit provides for a uniform rate at which grains imbibe water and plant additives from the surrounding environment that consequently results in a uniform seedling development rate. Achieving a uniform imbibition rate for grains improves the productivity, efficiency, and economics for industries that rely on grain germination and seedling development to produce a product.

Grains contemplated to be imbibed with the systems and methods of the present disclosure may include any vascular seed plant, and particularly angiosperms, which possess the specialized endosperm food supply inside the seed coat. Endosperm is the chief storage tissue in the seeds of cereal grains and grain legumes, which are both utilized as major food sources by humans and animals. Cereal grains may include, but are not limited to, wheat (Triticum aestivum), corn (Zea mays), rice (Oryza sativa), wild rice (Zizania palustris), barley (Hordeum vulgare), oats (Avena sativa), rye (Secale cereale), Sorghum (Sorghum bicolor), bulgur, teff Eragrostis tef), triticale (Triticosecale), and millet (Panicum millaceum). Other grains may include, but are not limited to, Amaranth, buckwheat (Fagopyrum esculentum), and quinoa (Chenopodium quinoa Willd.). Grain legumes, also known as pulses, may include, but are not limited to, soybean (Glycine max), lentil (Lens esculenta), peas (Pisum sativum), chick pea (Cicer arietinum), Faba bean (Vicia faba), cowpea (Vigna sinensis), pigeonpea (Cajanas cajan, Cajanus indicus), and peanut (Arachis hypogaea).

Plant additives contemplated to be imbibed into grains using the systems and methods of the present disclosure include, but are not limited to, nutrients, fungicides, insecticides, signaling compounds, and exogenous phytohormones. Exemplary exogenous phytohormones of the present disclosure include, but are not limited to, cytokinins for promoting cell division (e.g., synthetic cytokinin thidiazuron), auxins for promoting plant growth (e.g., synthetic auxin 1-Naphthaleneacetamide), and gibberellins for controlling seed germination (e.g., gibberellic acid (GA₃)).

While certain aspects of the present disclosure are shown and described herein, it is understood that such aspects are merely exemplary. The present disclosure is not intended to be limited to these specific aspects and may encompass other aspects or embodiments. Therefore, specific system and method details disclosed herein are not to be interpreted or inferred as limiting, but merely as a basis for the claims and as a representative basis for teaching one skilled in the art how to make and use the disclosed subject matter.

It must further be noted that the singular terms “a,” “an,” and “the” as used herein may include plural referents unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means.

All words of approximation as used in the present disclosure and claims should be construed to mean “approximate,” rather than “perfect” or “exact,” and may be used as a modifier to any other word, number, quantity, quality, value, or specified parameter. Words of approximation, include, but are not limited to terms such as “about,” “approximately,” “around,” “almost,” “generally,” “largely,” “essentially,” “substantially,” etc. As used herein, in some aspects, the terms “about” or “approximately” when preceding a numerical value may indicate the value plus or minus a range of 0.1, 0.2, 0.3, 0.4 or 0.5 inch of Hg. In other aspects, the terms “about” or “approximately” when preceding a numerical value may indicate the value plus or minus a range of 1, 2, 3, 4 or 5 inches of Hg. In further aspects, the terms “about” or “approximately” when preceding a numerical value may indicate the value plus or minus a range of 0.1, 0.2, 0.3, 0.4, or 0.5 ppm. In still further aspects, the terms “about” or “approximately” when preceding a numerical value may indicate the value plus or minus a range of 1, 2, 3, 4, or 5 ppm. In some aspects, the terms “about” or “approximately” when preceding a numerical value may indicate the value plus or minus a range of 0.1, 0.2, 0.3, 0.4, or 0.5 ml. In other aspects, the terms “about” or “approximately” when preceding a numerical value indicates the value plus or minus a range of 1, 2, 3, 4, or 5 ml. In further aspects, the terms “about” or “approximately” when preceding a numerical value indicates the value plus or minus a range of 1, 2, 3, 4, or 5 seconds. In still further aspects, the terms “about” or “approximately” when preceding a numerical value indicates the value plus or minus a range of 1, 2, 3, 4, or 5 minutes. In other aspects, the terms “about” or “approximately” when preceding a numerical value indicates the value plus or minus a range of 1, 2, 3, 4, or 5° Celsius. In further aspects, the terms “about” or “approximately” when preceding a numerical value indicates the value plus or minus a range of 1, 2, 3, 4, or 5 feet.

Furthermore the transitional phrase “comprising” that is synonymous with “including,” “containing,” and “characterized by” as used herein is inclusive or open-ended and does not exclude additional, unrecited elements, steps or ingredients. Alternatively the transitional phrase “consisting of” as used herein is closed and excludes any element, step or ingredient not specified. The transitional phrase “consisting essentially of” limits the scope of a claim to the specified materials or steps and those that do not materially affect the basic and novel characteristics of the claims.

For purposes of the present disclosure, the terms “negative pressure,” “reduced pressure,” and “vacuum pressure” are used interchangeably and defined herein as “pressure that is less than standard atmospheric pressure.” The term “positive pressure” is defined herein as “pressure that is greater than standard atmospheric pressure.” The term “standard atmospheric pressure” is defined herein as “29.92 inches of mercury (‘Hg’).”

FIG. 1 illustrates one aspect of the present disclosure, in particular, a system (10) for imbibing grains (12) using negative or reduced pressure to mitigate variations in water and plant additive imbibition rates that may negatively impact germination times and resultant seedling development. The system (10) may comprise a plurality of grains (12) to be imbibed, including at least one of cereal grains, grain legumes, and other grains for animal or human consumption. The grains (12) may be intended for use in commercial applications such as, but not limited to, the following industries:
Seed treatment applications including insecticides and fungicides prior to drying the grain for short term storage and transportation for the agronomic industries;
Seed priming applications to high value crops including seed treatment and biological introduction of plant additives to maximize individual seed response and yield production for the horticulture industries;
Seed priming applications to maximize germination rates, enzyme yield, and fermentable sugar yield in grain to provide a more efficient, profitable, and sustainable animal protein production for the animal feeding industries, whereby enteric methane emissions and carbon dioxide emissions from ruminants are reduced.
Seed priming applications to improve the rate and ratio at which input substrates are transformed into usable fermentation byproducts for the malting industries, whereby wastes associated with the fermentation process such as carbon dioxide and methane are reduced.

Shown in FIG. 1, the system (10) may further comprise an aqueous solution (14) for imbibing the grains (12). The solution (14) may comprise a sufficient amount of water (H₂O) for submerging the grains (12). As a non-limiting example, the water (H₂O) may be approximately 2,000 ml of sterilized distilled water (H₂O). It is contemplated by the present disclosure that greater amounts of water (H₂O) may be utilized for largescale, commercial operations.

The solution (14) may comprise approximately 3.0-10.0 ppm of plant additives. Plant additives contemplated to be imbibed into grains using the systems and methods of the present disclosure include, but are not limited to, nutrients, fungicides, insecticides, signaling compounds, and exogenous phytohormones. Exemplary exogenous phytohormones of the present disclosure include, but are not limited to, cytokinins for promoting cell division (e.g., synthetic cytokinin thidiazuron), auxins for promoting plant growth (e.g., synthetic auxin 1-Naphthaleneacetamide), and gibberellins for controlling seed germination (e.g., gibberellic acid (GA₃)). In some aspects, the solution (14) may comprise approximately 3.0-10.0 ppm of GA₃. In other aspects, the solution (14) may comprise approximately 0.75-10.0 ppm of GA₃, approximately 1.0-2.0 ppm of 1-Naphthaleneacetamide, and approximately 1.0-5.0 ppm of thidiazuron.

In some aspects, the solution (14) may optionally include approximately 40.0-60.0 ppm of a reactive oxygen species (“ROS”). ROS are a class of highly reactive and oxygen-bearing molecules that include superoxide anion (O₂^{·-}), hydrogen peroxide (H₂O₂), hydroxyl radical (OH), and singlet oxygen (¹O₂). It is well known that ROS plays an important role in the regulation of seed dormancy, germination, and deterioration. See Kurek et al., Reactive Oxygen Species as Potential Drivers of the Seed Aging Process, PLANTS, Vol. 8, Iss. 6, p. 174 (Jun. 14, 2019); see also Considine et al., Oxygen and reactive oxygen species-dependent regulation of plant growth and development, PLANT PHYSIOLOGY, Vol. 186, Iss. 1, pp. 79-92 (May 27, 2021). In particular, ROS may interact with the hard outer layer of the seed coat causing it to weaken and permit water to enter quickly to initiate germination. It is also well known that ROS may be used as a weapon against pathogens on the seed coat, being either directly toxic against pathogenic microorganisms or trigger hypersensitive reaction and programmed cell death at sites attacked by pathogens. See Lamb et al., The Oxidative Burst In Plant Disease Resistance, ANNUAL REVIEW OF PLANT BIOLOGY, Vol. 48, pp. 251-274 (June 1997). Therefore, utilizing ROS in the solution (14) will constitute a defensive reaction for the grains (12) against infection by harmful microorganisms.

Further shown in FIG. 1, the system (10) may comprise a vacuum chamber (16) for applying negative pressure to the grains (12) in the solution (14) for a set imbibition duration time. The vacuum chamber (16) may comprise a rigid enclosure from which air and other gases are removed by a vacuum pump. The vacuum chamber (16) is configured to create a low-pressure environment inside its chamber, commonly referred to as a vacuum. The vacuum chamber (16) may be comprised of stainless steel, aluminum, other metals, or rigid plastics. As a non-limiting example, the vacuum chamber (16) may include a 5.7 L enclosure connected to a 3.6 CFM single-stage vacuum pump. It is contemplated by the present disclosure that other vacuum chambers (16) of different sizes and strengths may also be utilized in the system (10) depending on the amount of grains (12) needed to be imbibed, such as for large scale operations depending on the industry.

Still further shown in FIG. 1, the system (10) may comprise a growing station (18) for cultivating the imbibed grains into plant seedlings. In some aspects, the growing station (18) may comprise soil cultivation such as in large scale outdoor agricultural production operations that involve extensive land, machinery, and labor resources to produce crops or raise livestock. In other aspects, the growing station (18) may comprise indoor and/or outdoor stations where grains are grown hydroponically or aeroponically using natural light, artificial light, or combinations thereof. While both hydroponics and aeroponics cultivate plants without soil, the techniques are distinct. In hydroponics plants are typically submerged in water that has been enriched with nutrients. Conversely in aeroponics, the roots of plants are suspended in the air and misted with water that has been enriched with nutrients. When compared to soil-cultivated plants, hydroponic and aeroponic growing stations provide improved growth, yield, quality, and production to the plant seedling. Aeroponics provide even greater benefits when compared to hydroponics, where plants grown aeroponically have 100% access to CO₂ for photosynthesis and consume 70% less water than hydroponics. See Kumar et al., Vertical farming and organic farming integration: a review, ORGANIC FARMING, 2^{nd} ED., pp. 291-315 (2023). Thus, in preferred aspects of the present disclosure the growing station (18) comprises aeroponics.

As a non-limiting example, the growing station (18) of the present disclosure may comprise a vertical series of platforms forming a tower structure. Each platform of the series may comprise a plurality of seed trays for housing the grains (12), wherein each seed tray may be approximately 8-12 feet in width and 10-14 feet in length. Each seed tray may be configured to house grains (12) placed therein at approximately 1-3 inches in thickness. Each platform of the tower may comprise a series of misting nozzles located above the seed trays, the misting nozzles designed to mist the grains (12) at set intervals with water that has been enriched with nutrients for optimal germination, uniformity, and growth of the plant seedlings. The growing station (18) may be a closed loop system that includes at least one storage tank used to store nutrient-rich water for application to the grains, in addition to conserving excess water after grain application, wherein the excess water may be later reapplied to the grains to reduce water consumption and improve efficiency of the growing station (18). The growing station (18) may be housed indoors or outdoors and use natural light, artificial light, or combinations thereof to initiate germination and encourage rapid growth of the plant seedlings for improved yields and performance. Once the plant seedling reaches a preferred stage of growth, the seed trays housing the grains may be removed from their respective platforms on the tower structure and the plant seedlings harvested for their intended use in the agronomic, horticulture, animal feed, and malting industries.

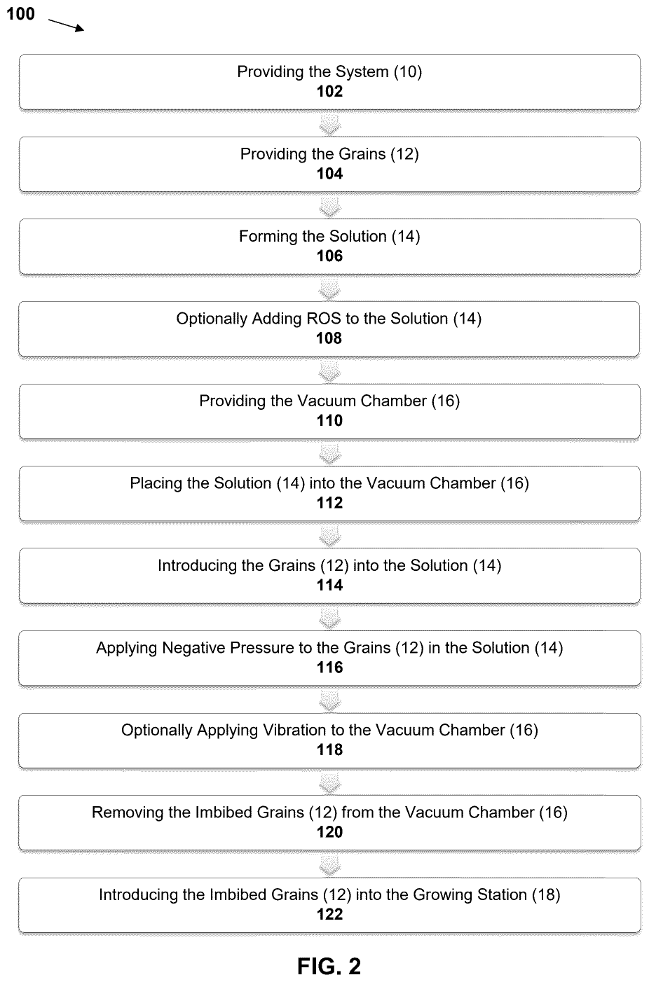

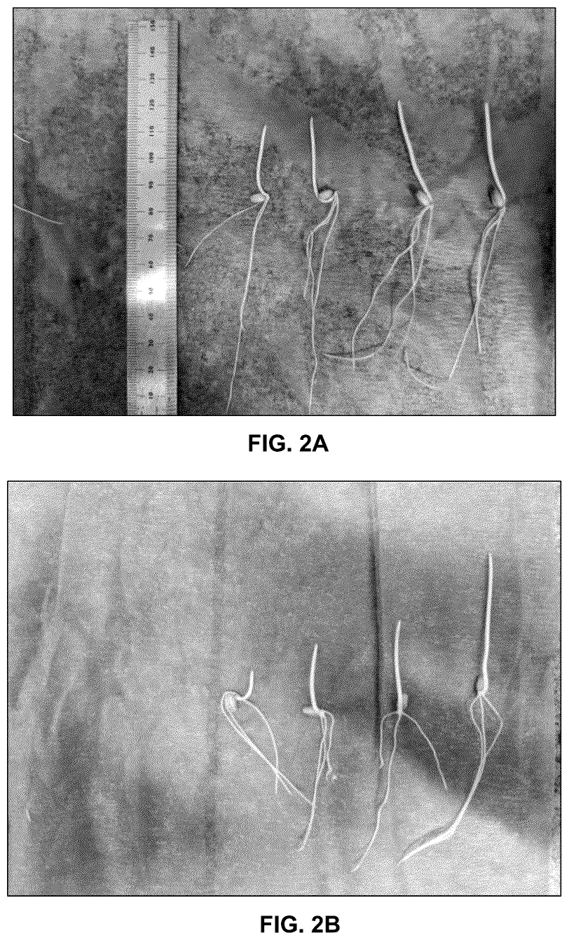

FIG. 2 illustrates another aspect of the present disclosure, in particular, a method (100) for imbibing grain using negative pressure to mitigate variations in water and plant additive imbibition rates that may negatively impact germination times and resultant seedling development. The method (100) may comprise providing (102) the system (10) of FIG. 1, along with the grains (12) for imbibition (104). The method (100) may further comprise forming (106) the solution (14). As a non-limiting example, the solution (14) may be formed by introducing approximately 3.0-10.0 ppm of plant additives into approximately 2,000 ml of sterilized distilled water (H₂O). It is contemplated by the present disclosure that greater amounts of water (H₂O) may be utilized for large-scale, commercial operations. As a non-limiting example, the plant additives may comprise at least one of the exogenous phytohormones cytokinin thidiazuron, auxin 1-Naphthaleneacetamide, and gibberellic acid (GA₃). In some aspects, the solution (14) may comprise approximately 3.0-10.0 ppm of GA₃. In other aspects, the solution (14) may comprise approximately 0.75-10.0 ppm of GA₃, approximately 1.0-2.0 ppm of 1-Naphthaleneacetamide, and approximately 1.0-5.0 ppm of thidiazuron. Optionally, the method (100) may comprise adding (108) approximately 40.0-60.0 ppm of an ROS to the solution (14), such as, but not limited to, hydrogen peroxide (H₂O₂). After introducing the plant additives, and optional ROS, into the sterilized distilled water (H2O), the solution (14) may be mixed and thereafter chilled to approximately 6°-18° Celsius.

Shown in FIG. 2, the method (100) may comprise providing (110) the vacuum chamber (16). The method (100) may further comprise placing (112) the solution (14) into the vacuum chamber (16), and thereafter introducing (114) the grains (12) into the solution (14). Using the vacuum chamber (16), negative pressure may be applied (116) to the grains (12) in the solution (14) at a certain pressure and for a set imbibition duration time to promote rapid and uniform rates of imbibition. As a non-limiting example, negative pressure may be applied (116) to the grains (12) in the solution (14) at approximately 10.0-25.0 inches of Hg for approximately 60-240 minutes. In some aspects, negative pressure may be constantly applied (116) to the grains (12) in the solution (14) at approximately 10.0 inches of Hg for a total imbibition duration time of approximately 180 minutes. In other aspects, negative pressure may be constantly applied (112) to the grains (12) in the solution (14) at approximately 20.0 inches of Hg for approximately 180 minutes. In other aspects, negative pressure may be constantly applied (112) to the grains (12) in the solution (14) at approximately 25.0 inches of Hg for approximately 180 minutes. In further aspects, negative pressure may be intermittently applied (116) to the grains (12) in the solution (14) at approximately 10.0 inches of Hg for a total imbibition duration time of approximately 180 minutes, wherein the negative pressure may be released for approximately twenty seconds at fifteen minute intervals during the total imbibition duration time. A minimum negative pressure of approximately 10.0 inches of Hg applied (116) to the grains (12) in the solution (14) for a minimum imbibition duration time of approximately sixty minutes is needed to realize positive effects in rapid and uniform rates of imbibition.

Further shown in FIG. 2, the method (100) may optionally comprise applying vibration (118) to the vacuum chamber (16) during imbibition of the grains (12) for a set time period. As a non-limiting example, vibration may be applied (118) to the vacuum chamber (16) during imbibition of the grains (12) for approximately one minute every fifteen minutes of the imbibition duration time. Alternatively, vibration may be constantly applied (118) to the vacuum chamber (16) during the total imbibition duration time. Vibration of the vacuum chamber (16) influences a seed coat's inherent surface tension and slightly hydrophobic nature to promote imbibition. Thus vibration of the vacuum chamber (16) may help mitigate variations in water and plant additive imbibition rates that may negatively impact germination times and resultant seedling development.

Still further shown in FIG. 2, after negative pressure has been applied (116) to the grains (12) in solution (14) for the set imbibition duration time (including the optional vibration application (114)), the grains (12) may be removed (120) from the vacuum chamber (16) and introduced (122) into the growing station (18). Once the grains germinate and reach a preferred stage of growth in plant seedling development, the plant seedlings may be removed from the growing station (18) and harvested for their intended use in the agronomic, horticulture, animal feed, and malting industries.

FIGS. 2A and 2B illustrate comparative examples showing the uniform and advanced development rate of seedlings for grains imbibed according to the method of FIG. 2 as contrasted to the variable and stunted development rate of seedlings for grains imbibed without negative pressure. In particular, FIG. 2A shows the development rate of seedlings after 72 hours in the growing station (18) for grains imbibed according to the method of FIG. 2. Grains (12) introduced in an aqueous solution (14) comprising approximately 3.0-10.0 ppm of GA₃ with negative pressure constantly applied (112) at approximately 25.0 inches of Hg for a total imbibition duration time of approximately 180 minutes was shown to reduce the variability in individual grain water uptake by 84% while improving the response to key phytohormones by over 25%. On the other hand, FIG. 2B shows the variable development rate of seedlings for grains after 72 hours in the growing station (18) for grains imbibed without negative pressure. As the contrasting pictorial representations demonstrate, the method of FIG. 2 provides for uniform seedling development to improve the productivity, efficiency, and economics for industries that rely on grain germination and plant seedlings to produce a product. The comparative results of seedling development shown in FIGS. 2A and 2B are further illustrated in Table 1 (below). In particular, Table 1 demonstrates less seedling variability in germination, coleoptile length, radicle length, and overall pixel area size when utilizing the method of FIG. 2A as compared to the control.

TABLE 1
Standard Deviation Comparison Across Control
and Negative Pressure Treatment.

| | Germination | Coleoptile | Radicle | Pixel Area |
|---|---|---|---|---|
| Treatment | (%) | (mm) | (mm) | (mm) |
| Negative | 0.9 | 5.1 | 9.2 | 1235 |
| Pressure | | | | |
| (FIG. 2A) | | | | |
| Control | 1.3 | 10.4 | 18.8 | 5782 |
| (FIG. 2B) | | | | |
| Delta | 31% | 51% | 51% | 79% |

### EXAMPLES

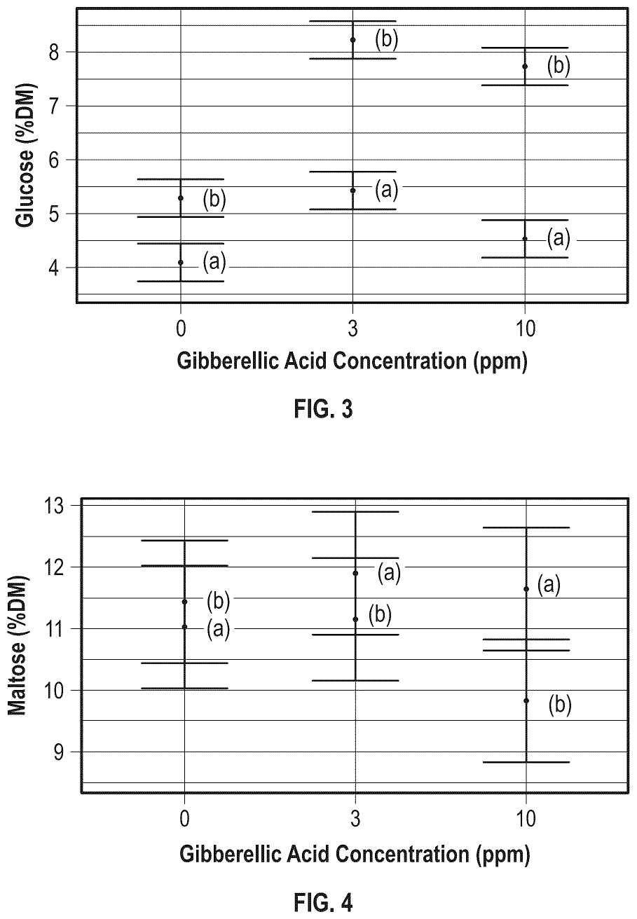

Illustrated in FIGS. 3-13, the following non-limiting examples demonstrate the combined positive influence of vacuum imbibition and the use of exogenous phytohormone introduction on grain germination and enzymatic activity of the present disclosure. Germination percentages were evaluated according to Association of Official Seed Analysts (“AOSA”) Standards, with grains evaluated at 96 hours for radicle and coleoptile emergence with replicated 15 g samples. Seedling composition was assessed through near infrared spectroscopy, and expressed as concentration on a dry weight basis, with enzymatic activity being indirectly assessed through glucose, maltose, fructose, and total fermentable sugar concentration on a dry matter basis. Vacuum was applied to a 5.7 L vacuum chamber connected to a 3.6 CFM single-stage vacuum pump. Moisture content was evaluated by surface drying approximate 15 g samples, after which the samples were dried in a forced air oven at 150° C., using the following formula:

$$Moisture \% = 1 - \frac{wet weight - dry weight}{wet weight}$$

In each example, 1,000 g samples of Winner hard red winter wheat (Triticum aestivum) were submerged in 2,000 ml of water and chilled to about 12° C. for 180 minutes under vacuum and phytohormone conditions described. Where indicated in the examples, vibration was applied to the vacuum chamber for about one minute approximately every fifteen minutes during imbibition, using a hand-held percussion massage gun held against the vacuum chamber. For the intermittent vacuum imbibition examples, the vacuum was released approximately every fifteen minutes during imbibition, and then returned to the original vacuum condition achieved in approximately twenty seconds.

### Example 1: Addition of Exogenous Gibberellic Acid (GA₃)

Demonstrated in FIG. 3, presented as least square mean estimates and 95% confidence intervals, it may be observed that 3.0 ppm of water-soluble gibberellic acid (GA₃) in sterilized distilled water (H₂O) under 25 in. Hg vacuum, significantly (p<0.05) improved germination percentage, glucose, fructose, and total fermentable sugar concentration and yield on a dry matter basis, while no significant difference was observed for maltose concentration. Additionally, 10.0 ppm of water-soluble gibberellic acid in water solution under 25 in. Hg vacuum, significantly (p<0.05) improved glucose and total fermentable sugar concentration and yield on a dry matter basis, while no significant difference was observed for maltose concentration, germination percentage, or fructose concentration.

### Example 2: Addition of Exogenous Gibberellic Acid (GA₃), Auxins, Cytokinins, and Hydrogen Peroxide (H₂O₂)

Demonstrated in FIGS. 8-12, which show the least square mean estimates and 95% confidence intervals of the data presented, it may be observed that the combination of exogenous phytohormones and negative pressure resulted in significant (p<0.05) increases in gemination percentage, coleoptile length, radicle length, and biomass pixel count when compared to non-vacuum treatments for gibberellin and cytokinin type hormones, while reducing development for auxin type hormones. This is expected due to the positive aspects of gibberellins and cytokinin for germination, and the negative influence of auxins especially at higher concentrations. The application of negative pressure hydration was observed to clearly increase the effectiveness of phytohormone application.

### Example 3: Effects of Application of Intermittent Vacuum and Vibration on Imbibition of Grains

FIG. 13 is presented as least square mean estimates and 95% confidence intervals. As demonstrated therein, alternating (i.e., applying and releasing) negative pressure significantly increased grain moisture percentage as compared with applying constant negative pressure, as did application of external vibration to the vacuum chamber. No difference was detected between alternating vibration treatments.

As demonstrated in the examples above, the impact of utilizing negative pressure during grain imbibition has provided beneficial results in reducing the variation in grain-to-grain seedling development. These results were surprising and unexpected, as it was assumed the application of negative pressure would have similar outcomes as the application of positive pressure. Results of experimentation with negative pressure and positive pressure are illustrated in FIGS. 14, 15.

Shown in FIG. 14, a pictorial representation of cross sections of grains is provided comparing endosperm hydration at varying levels of negative pressure, positive pressure, and a control. In particular, a series of experimental trials were conducted involving replicated samples of Winner hard red winter wheat grains. Treatment conditions for each experimental trial are summarized, below:
No Imbibition: No imbibition applied to grain. Neither negative pressure nor positive pressure applied to grain. Grain cut cross-sectionally with a scalpel.
Control: Grain placed in hydration vessel and imbibed in approximately 2,000 ml of sterilized distilled water (H₂O). Imbibition time period of approximately 240 minutes. Neither negative pressure nor positive pressure applied to grain during imbibition time period. After the imbibition time period, grain was removed from hydration vessel and cut cross-sectionally with a scalpel.
Negative Pressure: Grain placed in hydration vessel and imbibed in approximately 2,000 ml of sterilized distilled water (H₂O). Imbibition time period of approximately 240 minutes. Constant negative pressure of approximately 25 in. Hg applied to grain during imbibition time period. After the imbibition time period, grain was removed from hydration vessel and cut cross-sectionally with a scalpel.
Positive Pressure: Grain placed in hydration vessel and imbibed in approximately 2,000 ml of sterilized distilled water (H₂O). Imbibition time period of approximately 240 minutes. Constant positive pressure of approximately 163 in. Hg applied to grain during imbibition time period. After the imbibition time period, grain was removed from hydration vessel and cut cross-sectionally with a scalpel.

Further shown in FIG. 14, the applications of both positive pressure and negative pressure were observed to significantly increase the physical volume size of individual grains. This increase in grain volume size appeared to result from improved endosperm hydration as compared to the control. In light of these experimental trials, it was expected that the application of either positive pressure or negative pressure during imbibition would lead to similar outcomes in affecting germination rates, the mobilization of starches, and the development rate of seedlings. However as shown in FIG. 15, these expectations were unfounded as further experimentation led to surprising and unexpected results.

Shown in FIG. 15, a graph is provided comparing germination percentages for the grains of FIG. 14, namely, the germination percentages of grains imbibed with positive pressure, negative pressure, and the control using the treatment conditions set forth above. In particular, experimentation with the application of positive pressure during grain imbibition was found to significantly lower germination percentages as compared to the control. It was discovered that the application of positive pressure during imbibition causes physical damage to internal structures of the grain (e.g., interconnected tissues and cell walls). Such physical damage includes cytorrhysis, the permanent and irreparable damage to the cell wall due to loss of internal pressure within the cell. The integrity of cells within grains is dependent on a pressure differential between the cell and the surrounding environment. If the pressure differential becomes too great, the cell will collapse resulting in the destruction of the cell. Because intactness of cells within the grain is required for germination, therefore, the application of positive pressure was found to reduce germination rates, seedling development, and uniformity of the same.

The application of negative pressure during grain imbibition was expected to lead to similar results in reduced germination rates, seedling development, and uniformity. In particular, the application of negative pressure to an aqueous solution during imbibition was believed to cause degassing (i.e., the lowering of oxygen content in dissolved water through the escape of dissolved oxygen). Such degassing was expected to be determinantal to germination and seedling development rates due to the increased oxygen requirements of metabolically active grain embryos as they develop into seedlings. Surprisingly and unexpected, the application of negative pressure during grain imbibition appears to avoid undesirable detrimental effects to cellular integrity by maintaining the pressure gradient between the cell and the surrounding environment. In addition, as air is removed from internal compartments of the grain during negative pressure grain imbibition, water fills these compartments due to the inability of water to be compressed. Such replacement of air with water therefore prevents the collapse of the grain's cellular structures under increasing negative pressure, resulting in a healthy, viable, and hydrated grain. The optional introduction of ROS to the aqueous solution of the present disclosure was further found to increase the dissolved oxygen content and provide an anaerobic respiration substrate. Therefore the application of negative pressure during grain imbibition, in contrast to positive pressure and the control, resulted in improved germination rates, seedling development, and uniformity of the same that were surprising and unexpected.

The present disclosure is not to be limited to the particular aspects and examples described herein. In particular, the disclosure contemplates numerous variations in systems and methods for overcoming individual differences in grain morphology using negative pressure to achieve a uniform rate at which grains absorb water, nutrients, and additives from the surrounding environment to increase the productivity, efficiency, and economics of industries that rely on grain germination and seedling development to produce a product. The foregoing description has been presented for purposes of illustration and description. It is not intended to be an exhaustive list or limit any of the disclosure to the precise forms disclosed. It is contemplated that other alternatives or exemplary aspects are considered included in the invention. The description is merely examples of aspects, processes, or methods of the disclosure. It is understood that any other modifications, substitutions, and/or additions can be made, which are within the intended spirit and scope of the disclosure. What is claimed is:

1. A method for increasing the effects of phytohormones on grains, comprising: forming a solution having a chosen concentration of at least one phytohormone in water; contacting the grains with the solution;
reducing the pressure over the grains and the solution to a first selected value; and maintaining the reduced pressure for a first chosen time period.

2. The method of claim 1, wherein the grains are chosen from barley, wheat, corn, sorghum, rice, oats, rye, and triticale, and mixtures thereof.

3. The method of claim 2, wherein the grains comprise Winner hard red winter wheat seedlings.

4. The method of claim 1, wherein the phytohormones are chosen from gibberellins, auxins, cytokinins, abscisic acid, ethylene, and mixtures thereof.

5. The method of claim 4, wherein the cytokinins comprise thidiazuron.

6. The method of claim 4, wherein the auxins comprise 1-Naphthaleneacetamide.

7. The method of claim 4, wherein the gibberellins comprise gibberellic acid (GA₃).

8. The method of claim 1, wherein the reduced pressure is about 20 inches of Hg, and the first chosen time period is between about 150 min. and about 180 min.

9. The method of claim 1, further comprising the steps of:
increasing the pressure to atmospheric pressure after said step of maintaining the reduced pressure for a first chosen time period;
permitting the grains and the solution to remain at atmospheric pressure for a second chosen time period;
reducing the pressure over the grains and the solution to a second selected value; and maintaining the reduced pressure for a third chosen time period.

10. The method of claim 9, wherein the first selected value of reduced pressure is approximately equal to the second selected value of reduced pressure.

11. The method of claim 1, further comprising the step of vibrating the vacuum chamber at a chosen vibrational frequency during said step of maintaining the reduced pressure for a fourth chosen period of time.

12. A method for overcoming differences in grain morphology using negative pressure to achieve a uniform imbibition rate, comprising:
providing a system for imbibing grains using negative pressure, the system comprising
a) a plurality of grains to be imbibed;
b) an aqueous solution for imbibing the grains, the solution comprising:
i. water;
ii. approximately 3.0-10.0 ppm of plant additives; and
iii. approximately 40.0-60.0 ppm of a reactive oxygen species;
c) a vacuum chamber configured to apply negative pressure; and
d) a growing station for cultivating imbibed grains into plant seedlings;
placing the solution into the vacuum chamber;
introducing the grains into the solution;
reducing the pressure over the grains and the solution to a first selected value comprising approximately 10.0-25.0 inches of Hg;
maintaining the reduced pressure for a first chosen time period comprising approximately 60-240 minutes;
removing imbibed grains from the vacuum chamber and the solution;
introducing the imbibed grains into the growing station;
growing the grains in the growing station into plant seedlings; and
harvesting the plant seedlings.

13. The method of claim 12, wherein the grains are chosen from barley, wheat, corn, sorghum, rice, oats, rye, and triticale, and mixtures thereof.

14. The method of claim 12, wherein the plant additives comprise phytohormones chosen from gibberellins, auxins, cytokinins, abscisic acid, ethylene, and mixtures thereof.

15. The method of claim 14, wherein the cytokinins comprise thidiazuron.

16. The method of claim 14, wherein the auxins comprise 1-Naphthaleneacetamide.

17. The method of claim 14, wherein the gibberellins comprise gibberellic acid (GA₃)

18. The method of claim 12, further comprising the step of vibrating the vacuum chamber at a chosen vibrational frequency during said step of maintaining the reduced pressure for a first chosen time period.

19. The method of claim 12, further comprising the steps of:
increasing the pressure to atmospheric pressure after said step of maintaining the reduced pressure for a first chosen time period;
permitting the grains and the solution to remain at atmospheric pressure for a second chosen time period;
reducing the pressure over the grains and the solution to a second selected value; and maintaining the reduced pressure for a third chosen time period.

20. A system for imbibing grains using negative pressure to overcome differences in grain morphology to achieve a uniform imbibition rate, the system comprising:
a plurality of grains to be imbibed;
an aqueous solution for imbibing the grains, the solution comprising:
a) water;
b) approximately 3.0-10.0 ppm of plant additives; and
c) approximately 40.0-60.0 ppm of a reactive oxygen species;
a vacuum chamber configured to apply negative pressure to the grains in the aqueous solution; and
a growing station for cultivating imbibed grains into plant seedlings.
